(19) United States
(12) Patent Application Publication
(10) Pub. No.: US 2010/0155314 A1
(43) Pub. Date: Jun. 24, 2010

(54) Auto-injecting onsite dual tank microbial inoculator for use in in-situ bioremediation of FOG (Fats, Oils and Grease)

(76) Inventors: Markus Johannes Lenger, Waikoloa, HI (US); Robert Edwin Carr, Odessa, MO (US)

Correspondence Address: MARKUS JOHANNES LENGER, 68-1853 KAUPAPA WAY, WALKOLOA, HI, 96738, US

(21) Appl. No.: 12/344,189

(22) Filed: Dec. 24, 2008

(51) Int. Cl. C02F 11/02; C12M 1/36

(52) U.S. Cl. 210/104; 435/286.5

(57) ABSTRACT

A method to continuously inoculate (grow) microbial cultures on-site whereby a live culture of grease reducing microbes can be periodically injected directly into grease interceptors, grease traps and grease vaults. The system consists of a two tank system, one a isolated microbial storage tank (1) and two a inoculation tank (2). Microbial concentrate is periodically injected into the inoculation tank (2), filtered water is added and aerated (10) allowing the microbes to wake up into a vegetative state. Inoculation time is set for optimal growth and nutrition absorption time. The finished inoculated and vegetative microbial cultures are then injected into grease interceptors, grease traps and grease vaults containing a FOG (Fats, Oils and Grease). This process is continuously repeated to ensure viable microbial cultures for the bioremediation of FOG.

### CROSS-REFERENCE TO RELATED APPLICATIONS

This application claims the benefit of provisional patent application Ser. No. 60/008,710 filed Dec. 24, 2007 by the present inventors.

### FEDERALLY SPONSORED RESEARCH

Not Applicable

### SEQUENCE LISTING OR PROGRAM

Not Applicable

### BACKGROUND

1. Field of Invention

This invention relates to wastewater and solid treatment, specifically the bioremediation of Grease and Biosolids.

We have invented a method to continuously inoculate (grow) microbial cultures on-site whereby a live culture of grease reducing microbes can be periodically injected directly into mainly grease interceptors, grease traps and grease vaults, but also animal waste lagoons and waster water treatment plants. Although this invention is perfectly capable of being used as a stand-alone embodiment, it was tested non-commercially over the last 4 years together with a device for in-situ bioremediation of liquid waste (PTO provisional patent application 60/760,458 with filing date of Jan. 20, 2006, PTO Ser. No. 11/779,841 with filing date of Jul. 18, 2007) to establish reducing it to practical application. The details of the system has neither been published, shown to the public or sold as of this filling.

2. Prior Art

US Patent 2003/0008382 (2003) to Tisinger, et al. discloses that bioaugmentation can be added by a liquid metering pump drawing on a container that is replenished on a periodic basis (0008). Gram-positive micro organism, Bacillus megaterium, can effectively and efficiently degrade fats, oils and grease (as taken from the abstract) The use of a 2 tank system to animate the vegetative microbes is not discussed.

U.S. Pat. No. 5,840,182 (1998) to Lucido, et al. discloses that the output port on a pump is operably connected to a drain or trap for discharging a preselected amount of the contents of a bioreactor chamber containing water, nutrients and microorganisms (col. 8, lines 26-31) (See FIG. 1 and FIG. 6). Air is supplied to the bioreactor by a air-pump (col. 7, lines 40-48). A mixer such as a stirrer or paddle can be operably installed in the bioreactor chamber (col. 10, lines 25-29)(see also U.S. Pat. Nos. 6,573,085; 6,402,941; and 6,488,852) Lucido, et al. also describe, as background, the use of structures to support microbial films (See column 2, lines 1-16) The use of a 2 tank system to animate the vegetative microbes is not discussed.

U.S. Pat. No. 6,187,193 (2001) to Ozama discloses that air is replenished from the lid, drain inlet and drain exit of the grease trap, and constantly supplied into the drainage surface by sprinkling the drainage. (col. 4, lines 23-25) An impeller installed in the grease trap rotates to effect the stirring and splashing to activate the aerobic microorganisms (col. 4, lines 35-38). Ozama does not describe the periodic replenishing of microbes into the trap nor the use of a biofilm. He uses a pump to agitate the liquid and relies on sprinkling for air supply. The use of a 2 tank system to animate the vegetative microbes is not discussed.

U.S. Pat. No. 6,335,191 (2002) to Kiplinger, et al. describe a fairly complicated system comprising a bioreactor in which microbes are cultivated to be periodically released into grease traps (See column 3, lines 21-59). The use of a 2 tank system to animate the vegetative microbes is not discussed.

### DETAILED DESCRIPTION—FIG. 1—ILLUSTRATED EMBODIMENT

### 1. Inoculation Tank 1 (1)

The first tank of the 2 tank system is the microbial/nutrient mix holding tank (1). It is designed to hold the suspended cultures and all necessary nutrients until replenishment is needed. A small amount of microbial concentrate from inoculation tank 1, is periodically transferred to inoculation tank 2 (2). Therefore, the size of inoculation tank 1 establishes the maintenance and service cycle, since the system only needs to be accessed to replenish the microbial tank. In colder climates, it may be heated to maintain a favorable temperature of 20-30° C. An aeration rod (10) may be added if the microbes are stabilized using the common low PH method. For ionic (electrically charged) stabilization, no aeration rod is needed, since it would bring the cultures out of suspension prematurely. A high level (8) and low level switch (7) tell the control system when the tank has been replenished and when it is empty, triggering a replenishment request via the control system (6). If ionic (electrically charged) stabilized culture blends are used, inoculation tank 1 simply becomes a product holding tank. Tank 1 may be in the form of a product packaging such as a ready bladder container or canister or bottle. In this case it would be simply hooked up to the microbial transfer pump (3). A level sensor may be inserted but it is not necessarily needed, since the replenished amount or size of attached bottle/container is known to the controller/timer (6).

### 1. Inoculation Tank 2 (2)

The second tank of the 2 tank system is the microbial inoculation tank (2). It is supplied with oxygen via an aeration rod (10) and heated in colder climates to maintain a favorable temperature of 20-30° C. It periodically receives (typically every 24 hours) a small amount of microbial concentrate from the inoculation tank 1 (1) by means of the microbial transfer pump (3). It is then mixed with filtered (whereby chlorine is removed) tap water (11), by filling up the inoculation tank 2 until the high level switch (7) triggers a shut off of the fill valve (9). The controller/timer (6) aerates the microbial-nutrient-water mixture, not only bringing the cultures out of stasis, but also enabling an accelerated growth of the mixture. Since microbial cultures can reproduce every 19 minutes if conditions are perfect, even in this inoculation system, flourishing cultures of fully animated microbes can be achieved in 12 to 16 hours. Once the microbes are ready, they are injected via an injector pump (4) into either the grease interceptor directly (or animal lagoon, waste water treatment basin) or into the kitchen drain, having the added advantage to also treat (keep clean) the kitchen drain pipes, thus also eliminating the “jetting of the lines”. This would necessitate either locating the inoculator system near the kitchen, or use of an injector line leading into the kitchen drain or a pipe or ventilation stack that has access or at least drains into the kitchen drain line draining (usually but not necessarily) to the grease interceptor. A low level sensor (8) confirms to the controller (6) when the tank is empty, to shut off the injection pump (4) and to generate a time stamp and treatment record stored in the controllers (6) memory.

### 3. Transfer Pump (3)

The transfer pump (3) is used to transfer the microbial liquid from tank 1 (1) to tank 2 (2) at a given time interval triggered by the controller/timer (6).

### 4. Injector Pump (4)

The injector pump (4) is used to transfer the microbial liquid from tank 2 (2) to either the grease interceptor (or animal lagoon, waste water treatment basin) or directly into the kitchen drain by a given time interval triggered by the controller/timer (6).

### 5. Aerator System (5) (10)

The aerator system (5) (10) consists of an air pump (5) and aeration rods (10). It supplies air (oxygen) to either one or both tanks to supply the microbes with the necessary oxygen to feed their metabolism. It is turned on and off as needed by the control system (6).

### 6. Control System/Timer (6)

The control system/Timer (6) in its simplest form can be a multi channel timer or can be far more intelligent. It can either run on preprogrammed timer cycles such as 24 hours or can be triggered by an adaptive control adjusting the time intervals and spans based on the loading of the grease interceptor (or animal lagoon, waste water treatment basin). It can also be triggered via remote process or a switch pressed by a kitchen staff or engineer or even a supervisory agency or a contracting firm/consultant/inspector.

### 7. Low Level (7) and High Level (8) Switches

These switches simply sense the liquid level—e.g. relaying information whether they are submerged in liquid or not. This status is relayed to the controller (6) or any other monitoring system attached to it.

### 8. Fill Valve (9) and Water Filter (11)

The water filter (11) is hooked up to a municipal (or well water) supply and has the function to remove the chlorine, which would negatively affect the growth of the microbial cultures. The fill valve simply opens via a signal from the controller/timer (6) to fill the inoculator tank 2 (2). The high level switch (7) triggers via the controller/timer (6) that the tank is full and shuts off the fill valve (9), thus stopping the water flow into tank 2 (2). This will conclude the periodic replenishment cycle.

### DRAWINGS

I have included one drawing sheet.

FIG. 1: A schematic diagram of all the components of the inoculator system

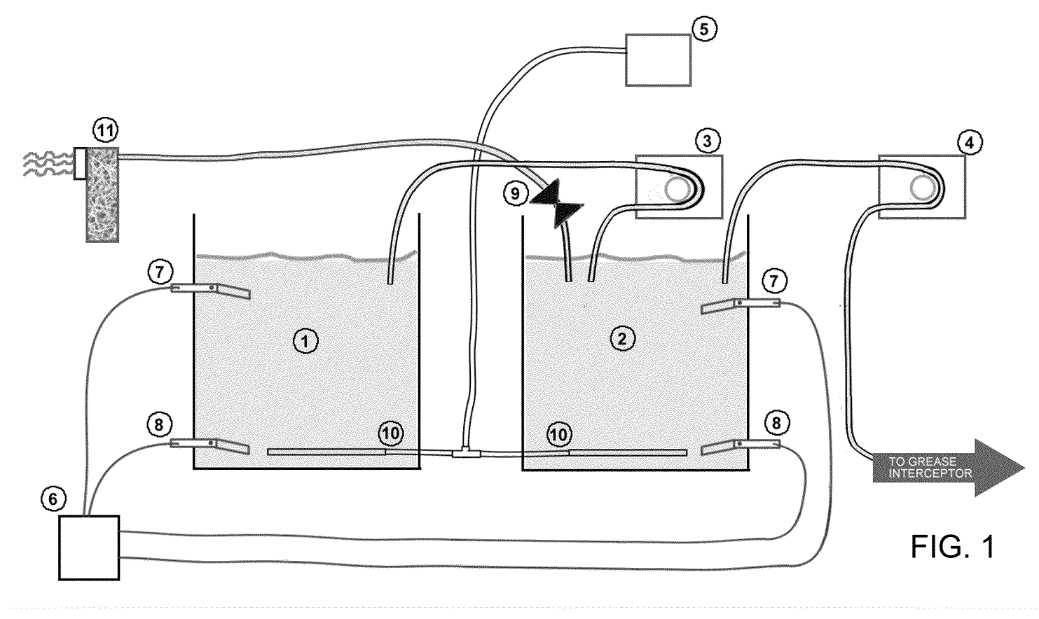

### DRAWINGS—REFERENCE NUMERALS

Inoculation tank 1 (1)
Inoculation tank 2 (2)
Microbial transfer pump (3)
Injector pump (4)
Air pump (5)
Control system/Timer (6)
High level switch (7)
Low level switch (8)
Fill Valve (9)
Aeration rod (10)
Water filter (11)

Advantages

Grease interceptors, grease traps and grease vaults (but also animal waste lagoons and waster water treatment plants) are most effectively treated with the use of microbial cultures commonly known as bioremediation. Grease interceptors, grease traps and grease vaults in particular, have a very short retention time (the time the waste sits in the chamber and thus is available for the application of a treatment) due to its sizing requirements (30 minutes at peak flow). Basically, it can be seen as a slow moving river, having a finite time available in which the biology can act upon it. This necessitates microbial cultures which are no longer in a vegetative state (one of suspension or slumber) but fully animated (awake) and ready to digest the grease. Since it takes 12 hours for microbial cultures to awake from the suspension, the cultures would come to life when they were already well inside the sewer system or the treatment plant, thus being entirely ineffective inside a grease interceptor or grease trap, since they would already have been flushed down the sewer line. While a vegetative (suspended) state of the cultures is necessary to achieve an acceptable shelf live and be stable enough for storage and transport, it also renders their automated periodical use ineffective in any application with a very short retention time or large flow volume. We therefore devised a 2 tank system to enable the microbial cultures to drop out of suspension, consume their nutrients and be ready for injection into a grease interceptor or other short retention time/high flow application.

Conclusions, Ramifications, and Scope

Thus the reader will see that at least one embodiment of our system provides a continuous source of ready, vegetative microbes available for the bioremediation of FOG in Grease interceptors, grease traps and grease vaults. This periodic replenishment of microbial cultures allows to maintain continuous bioremediation, even if harsh, antiseptic or toxic chemicals or cleaners are disposed of in the FSE (Food Service Establishment) and enter the Grease interceptors, grease traps or grease vaults.

While my above description contains many specificities, these should not be construed as limitations on the scope of the invention, but rather as an exemplification of on [or several] preferred embodiment thereof. Many other variations are possible. For example the same method can be used in Waste water treatment plants, animal waste lagoons such as hog and dairy lagoons. It may be used to amend irrigation water in agricultural application or any industrial process needing rapid culturing and deployment of microbial cultures.

Thus the scope of the embodiment should be determined by the appended claims and their legal equivalents, rather than by the example given.

We claim:

1. A unique and controllable microbial inoculator to promote acclimation towards a vegetative growth phase comprising of the following elements:
(a) a stabilized culture tank containing a selected microbial inoculant
(b) a controllable transfer method to inject a specified volume of inoculant into a second inoculation tank
(c) a second tank with a controllable aeration supply a redundant liquid level sensor enabling lower and upper level reading,
(d) a second tank with a controllable time cycle 5] second tank serving as a acclimation and adaption vessel.
(e) a second tank serving as a acclimation,adaption and/or fermentation vessel
(f) a second tank connected to a controllable transfer method towards a desired delivery destination.

2. The system of claim 1 wherein said elements are installed inside a grease interceptor, vault or other waste collecting enclosure.

3. The system of claim 1 wherein said elements are connected to a central control/telemetry unit.
whereby the system will inoculate vegetative microbes periodically and inject into grease interceptors, grease traps and grease vaults to enable, optimize and control bioremediation processes of FOG (Fats, Oils and Grease).
